(19) United States
(12) Patent Application Publication
(10) Pub. No.: US 2023/0071924 A1
(43) Pub. Date: Mar. 9, 2023

(54) PROBIOTIC COMPOSITION TO REDUCE PATHOGENESIS IN POULTRY AND METHODS FOR USE THEREOF

(71) Applicant: GINA SLOAN, STATESVILLE, NC (US)

(72) Inventor: GINA SLOAN, STATESVILLE, NC (US)

(21) Appl. No.: 17/818,591

(22) Filed: Aug. 9, 2022

Related U.S. Application Data
(63) Continuation-in-part of application No. 17/818,182, filed on Aug. 8, 2022.
(60) Provisional application No. 63/236,399, filed on Aug. 24, 2021.

(51) Int. Cl. A61K 35/745; A61K 35/747; A61K 38/47; A61K 38/46; A61K 31/717; A61K 31/719; A61K 31/7016; A61K 31/715; A61K 31/7032; A61K 47/14; A61K 47/12; A61K 47/18; A61K 47/24; A61P 1/14; A61K 9/00; A61P 31/04; A23K 10/18; A23K 20/189; A23K 50/75

(52) U.S. Cl. CPC A61K 35/745; A61K 35/747; A61K 38/47; A61K 38/465; A61K 31/717; A61K 31/719; A61K 31/7016; A61K 31/715; A61K 31/7032; A61K 47/14; A61K 47/12; A61K 47/183; A61K 47/24; A61P 1/14; A61K 9/0053; A61P 31/04; A23K 10/18; A23K 20/189; A23K 50/75; A61K 2035/115

(57) ABSTRACT

A probiotic composition comprised of a probiotic, a prebiotic, a complex-starch reducing enzyme, an enzymatic co-factor, and a pH modifier. The probiotic composition facilitates the creation of a biofilm within the GI tract of poultry that outcompetes and excludes pathogens. The probiotic composition can be incorporated into an eggwash and sprayed onto an egg, the sprayed egg developing a stable probiotic population within the GI tract of the pre-hatched chick. The probiotic composition can also be incorporated into poultry feed by applying the probiotic composition as a mash prior to pelleting. The probiotic composition can also be applied post-pelleting as a wet or dry spray, directly onto the pellets. In some instances, the probiotic composition can be added a drinking water source used by poultry as a water additive. Poultry that consume feed or water that contain the probiotic composition develop a stable probiotic population within their GI tracts.

This is a continuation-in-part of and claims benefits under pending prior application Ser. No. 17/818,182 filed 8 Aug. 2022, entitled “Probiotic Egg Wash”, which is a non-provisional patent application claiming all benefits under 35 U.S.C. § 119(e) of pending U.S. provisional patent application Ser. No. 63/236,399 filed 24 Aug. 2021, entitled “Probiotic Egg Wash”, in the United States Patent and Trademark Office, each of which are incorporated by reference in their entirety herein.

### FIELD OF THE INVENTION

The disclosure herein pertains to probiotic treatments generally, and particularly pertains to a probiotic composition that promotes the growth of probiotics and provides precursors for the outcompetition of potential pathogens and may be incorporated into poultry sustenance and/or an eggwash.

### DESCRIPTION OF THE PRIOR ART AND OBJECTIVES OF THE INVENTION

The U.S. Center for Disease Control (CDC) estimates that food-borne Salmonella causes 1.3 million infections every year in the United States (see: Prevention CDC and Salmonella at https://www.cdc.gov/salmonella/index.html). While there have been numerous attempts over the last decade to reduce the incidence of Salmonella, it continues to be a pathogen of concern, with only a 5% decrease in infection rates since 2016 (see: Tack D M, Marder E P, Griffin P M, et al. Preliminary incidence and trends of infections with pathogens transmitted commonly through food Foodborne Diseases Active Surveillance Network, 10 U.S. sites, 2015-2018. Am J Transplant. 2019; 19(6):1859-1863. doi:10.1111/ajt.15412). The Interagency Food Safety Analytics Collaboration attributes 14% of foodborne Salmonella to result from either chicken or turkey.

Salmonella is mainly a post-harvest concern for human disease and prevention. Therefore, in the poultry industry, Salmonella is controlled throughout the poultry life cycle but it has minimal impacts on the health of the chicken. Before post-harvest problems are optimized, the industry must hatch and grow a healthy chick. This hatch and grow out period can be impeded by gram negative and gram positive pathogens. In fact, there are many avian pathogens that can reduce the weight of chicks, such as avian pathogenic Escherichia coli (APEC) and Enterococcus cecorum, resulting in multi-million dollar loses annually to the poultry industry. APEC alone accounts for up to a 20% mortality rate during the poultry grow cycle. As an emerging poultry pathogen, E. cecorum is growing in incidence and severity with an 8% mortality rate. Despite current industry interventions these pathogens are still prevalent in many flocks. To date there are limited interventions that simultaneously decrease the number of gram negative and gram positive pathogens.

It is the objective of the present disclosure to provide an industry intervention that can reduce the carriage of poultry GI track pathogens such as those mentioned above to provide healthier chicks with lower morbidity and mortality rates throughout the grow out period. This objective is achieved by combining novel bacterial species with prebiotics and enzymes. This combination provides powerful out competition within the poultry GI track by establishing and maintaining a healthy microbiome.

It is another objective of the present disclosure to provide a probiotic composition that may be incorporated into a probiotic feed or probiotic eggwash.

It is another objective of the present disclosure to provide a probiotic composition that is formed from a mixture of probiotics including but not limited to: Aspergillus niger, Aspergillus oryzae, Bacillus amyloliquefaciens, Bacillus coagulens, Bacillus lentus, Bacillus pumilus, Bacteroides amylophilus, Bacteroides capillosus, Bacteroides ruminocola, Bacteroides suis, Bifidobacterium adolescentis, Bifidobacterium animalis, Bifidobacterium infantis, enterococcus cremorus, Enterococcus diacetylactis, Enterococcus faecium, Enterococcus intermedius, Enterococcus lactis, Enterococcus thermophilus, Lactobacillus animalis, Lactobacillus brevis, Lactobacillus bulgaricus, Lactobacillus cellobiosus, Lactobacillus curvatus, Lactobacillus delbruecki, Lactbacillus fermentum, Lactobacillus helveticus, Lactobacillus reuteri, Leuconostic mesenteroides, Pediococcus acidilactici, Pediococcus damnosus, Pediococcus pentosaceus, Propiniobacterium freudenreichii, Propiniobacterium shermanii, Rhodopseudomonas palustris, Saccharomyces cerevisiae, Lactobcillus casei, Lactobacillus rhamnosus, Lactobacillus plantarum, Lactobacillus paracasei, Lactobacillus acidophilus, Lactobacillus lactis, Bacillus subtilis, Bacillus lichenformis, Bifidobacterium bifidum, Bifidobacterium lactis, Bifidobacterium longum, and Bifidobacterium thermophilum. A preferable composition contains at least L. casei, L. rhamnosus, L. planitarum, L. paracasei, or B. lactis.

It is still another objective of the present disclosure to provide a probiotic composition formed from a mixture of probiotics with prebiotics including but not limited to: fructooligosaccharides (derived from natural or synthetic source such as onions, chicory, asparagus, artichokes, etc.), xylooligosaccharides (derived from natural or synthetic sources such as sugar cane, bamboo, soybeans and other vegetables as well as fruits), and galactooligosaccharides (derived from natural or synthetic sources such as legumes, dairy products, or root vegetables). A preferable composition contains at least xylooligosaccharide.

While the inclusion of probiotics and non-digestible fibers provide a healthy GI tract and microbiome, the bioavailability of alternate nutrients also plays a pivotal role in pathogen prevention. Therefore, it is still another objective of the present disclosure to provide a probiotic poultry feed formed from a mixture of probiotics with enzymes. The enzymes convert plant fibers and starches into digestible sugars and proteins that promote a healthy GI tract. Enzymes capable of digested grains such as corn, wheat, barley, and soybean include but not limited to xylase, glucanse, cellulase, xyloglucanse, fructose, galatosidase, esterase, mannase, and proteases. These enzymes can be isolated from numerous natural sources or created synthetically. Most notable, the mesophilic, filamentous fungal species Trichoderma reesei produces over 22 different enzymes within the above mentioned categories, resulting in extracts that are able to provide efficient enzyme activity against the above mentioned grains.

Therefore, it is still another objective of the present disclosure to provide a probiotic composition that includes a mixture of probiotics with enzymes. The enzymes convert fibers and starches into digestible sugars and proteins that promote a healthy GI tract. Enzymes capable of digested grains such as corn, wheat, barley, and soybean include but not limited to xylase, glucanse, cellulase, xyloglucanse, fructose, galatosidase, esterase, mannase, and proteases. These enzymes may be isolated from numerous natural sources or created synthetically. Most notable, the mesophilic, filamentous fungal species Trichoderma reesei produces over 22 different enzymes within the above mentioned categories, resulting in extracts that are able to provide efficient enzyme activity against the above mentioned grains. These enzymes may be included in the eggwash or feed pellets to prime the GI track for optimal use of the first meal and nutrients.

The availability of nutrients from complex starches that exist in plant-based diets is limited. These starches must first be broken down by enzymes to release the nutrients and create optimal growth conditions within the GI tract. Enzymes are one way to reduce complex starches into available nutrients that can be further processed by bacteria into antimicrobial compounds. As such the formulation may contain enzymes such as α-amylase, β-amylase, esterase, α-galactosidase, β-glucosidase, hemicellulose, invertase, lactase, β-mannase, pectinase, pullulanase, xylase, cellulase, xylosidase, β-glucanase, xyloglucanase, lipase, phytase, or a mixture of enzymes to release entrapped energy and nutrients. One example of a mixture of enzymes are those recovered from the growth of the fungal species Trichoderma reesei. T reesei is a filamentous fungal species with the capability of producing over 20 different lignocellulitic enzymes at a given time.

It is another objective of the present disclosure to provide a method of incorporating the probiotic composition into an eggwash that is to be applied to egg prior to hatching.

It is still another objective of the present disclosure to provide a method of incorporating the probiotic composition into a drinking water source as a water additive.

It is a further objective of the present disclosure to provide a method of incorporating the probiotic composition into feed pellets.

It is yet another objective of the present disclosure to provide a premix of the above components to be easily applied during the manufacture of livestock feed.

It is a further objective of the present disclosure to provide a final feed composition containing probiotics, prebiotics, and enzymes.

It is still a further objective of the present disclosure to provide a blend of the above components into a feed through a post manufacture application as a liquid.

It is still a further objective of the present disclosure to provide a blend of the above components into a feed through a post manufacture application as a powder.

Various other objectives and advantages of the present disclosure will become apparent to those skilled in the art as a more detailed description is set forth below.

### SUMMARY OF THE INVENTION

The aforesaid and other objectives are realized by providing a probiotic composition for the feed of livestock animals, such as poultry, resulting in an established GI tract microbiome that promotes health and outcompetes potential pathogens. The probiotic composition may comprise a probiotic mixture, a prebiotic, a complex-starch reducing enzyme, an enzymatic co-factor, and a pH modifier. The probiotic mixture includes a gastric-juice resistant probiotic and two heat-resistant probiotics. In a preferred embodiment, the probiotic feed is composed at least of L. casei, L. rhamnosus, L. planitarum, L. paracasei, and/or B. lactis at a concentration of between 10² to 10¹² Colony Forming Units (CFU) per mL in combination with either one prebiotic or a T. reesei derived enzyme mixture or a combination of all three components. Various application methods utilizing the probiotic composition to prevent pathogen growth in poultry GI tracts are considered within the scope of the present disclosure. The probiotic composition may be incorporated into the GI tracts of poultry by including the probiotic composition in food or water ingested by the poultry or directly spraying the poultry. The probiotic composition may also be incorporated into the GI tracts of poultry by applying the probiotic composition as an eggwash to eggs prior to or during incubation.

### BRIEF DESCRIPTION OF THE DRAWINGS

FIG. 1 shows a schematic representation of an application method of the probiotic composition as disclosed herein.

FIG. 2 shows an inhibitory effect of potential probiotic strains against E. coli, S. typhimirium, and L. monoctyogenes in Table 1 showing MIC in MRS.

FIG. 3 shows the survival rate of strains subjected to Acid and Bile in Table 2.

FIG. 4 demonstrates a graph of planktonic outcompetition.

FIG. 5 demonstrates a graph of biofilm exclusion.

FIG. 6 demonstrates a graph of feed composition and outcompetition.

FIG. 7 demonstrates a graph of feed composition without probiotics impacts the ability of pathogen growth.

FIG. 8 demonstrates a graph of feed composition with probiotics to enhance the ability to reduce pathogens.

FIG. 9 demonstrates a graph of feed composition with probiotics to promote successful outcompetition of gram positive species.

FIG. 10 demonstrates a table treatment group of eggs and type of treatment.

FIG. 11 demonstrates the table treatment group of eggs of FIG. 10 after an incubation period recording the number of villi per Field of View.

### DETAILED DESCRIPTION OF THE PREFERRED EMBODIMENT AND OPERATION OF THE INVENTION

Various exemplary embodiments of the present disclosure are described below. Use of the term “exemplary” means illustrative or by way of example only, and any reference herein to “the invention” is not intended to restrict or limit the invention to exact features or step of any one or more of the exemplary embodiments disclosed in the present specification. References to “exemplary embodiment”, “one embodiment”, “an embodiment”, “various embodiments”, and the like may indicate that the embodiment(s) of the invention so described may include a particular feature, structure, or characteristic, but not every embodiment necessarily incudes the particular feature, structure, or characteristic. Further, repeated use of the phrase “in one embodiment”, “in an exemplary embodiment”, or “in an alternative embodiment” do not necessarily refer to the same embodiment, although they may.

It is also noted that terms like “preferably”, “commonly”, and “typically” are not utilized herein to limit the scope of the invention or to imply that certain features are critical, essential, or even important to the structure or function of the invention. Rather, these terms are merely intended to highlight alternative or additional features that may or may not be utilized in a particular embodiment of the present disclosure.

The present disclosure is described more fully hereinafter with reference to the accompanying figures, in which one or more exemplary embodiments of the disclosure are shown. Like numbers used herein refer to like elements throughout. The disclosure may, however, be embodied in many different forms and should not be construed as limited to the embodiments set forth herein; rather, these embodiments are provided so that this disclosure will be operative, enabling, and complete. Accordingly, the particular arrangements disclosed are meant to be illustrative only and not limited as to the scope of the disclosure, and any and all equivalents thereof. Moreover, many embodiments such as adaptations, variations, modifications, and equivalent arrangements will be implicitly disclosed by the embodiments described herein and fall within the scope of the instant disclosure.

Although specific terms are employed herein, they are used in a generic and descriptive sense only and not for the purposes of limitation. Unless otherwise expressly defined herein, such terms are intended to be given their broad, ordinary, and customary meaning not inconsistent with that applicable in the relevant industry ad without restriction to any specific embodiment hereinafter described. As used herein, the article “a” is intended to include one or more items. Where only one item is intended, the terms “one and only one”, “single”, or similar language is used. When used herein to join a list of items, the term “or” denotes at least one of the items but does not exclude a plurality of items of the list.

For exemplary methods or processes of the disclosure, the sequence and/or arrangement of steps described herein are illustrative and not restrictive. Accordingly, it should be understood that, although steps of various processes or methods may be shown and described as being in a sequence or temporal arrangement, the steps of any such processes or methods are not limited to being carried out in any particular sequence or arrangement, absent an indication otherwise. Indeed, the steps in such processes or methods generally may be carried out in various different sequences and arrangements while still falling within the scope of the present disclosure.

Additionally, any references to advantages, benefits, unexpected results, or operability of the present disclosure are not intended as an affirmation that the disclosure has previously been reduced to practice or that any testing has been performed. Likewise, unless stated otherwise, use of verbs in the past tense (present perfect or preterit) is not intended to indicate or imply that the disclosure has previously been reduced to practice or that any testing has been performed.

As used herein, “probiotics” are bacteria that are known to be capable of adhering to intestinal cells and of excluding pathogenic bacteria on intestinal cells. To have this activity, the probiotic bacteria must remain viable in the product until it is consumed.

As used herein, “prebiotic” means food substances that promote the growth of probiotics in the GI tract. They are not usually broken down in the stomach and/or upper intestine or absorbed in the GI tract of the animal ingesting them, but they are fermented by the gastrointestinal microflora and/or by probiotics. The prebiotics that may be used in accordance with the present disclosure are not particularly limited and include all food substances known by those skilled in the art that promote the growth of probiotics in the intestines.

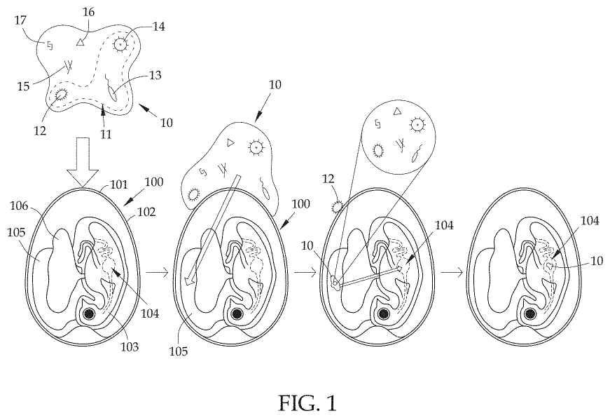

For a better understanding of the disclosure and its operation, turning now to FIG. 1, a probiotic composition 10 is disclosed that is configured to promote GI tract health and outcompete poultry pathogens. It will be understood that the probiotic composition 10 may be incorporated into the GI tracts of poultry by integrating the probiotic composition 10, in powderized or liquid form, into food (such as feed pellets) and/or water ingested by the poultry, and it may also be incorporated in the GI tracts by applying the probiotic composition 10 as an eggwash to eggs prior to or during incubation. The probiotic composition 10 comprises a probiotic mixture 11. All probiotic micro-organisms may be used for the purposes of the present disclosure. The poultry industry faces numerous pathogens of concern. One use of the probiotic composition 10 is the reduction of carriage and symptoms caused by multiple organisms including but not limited to Salmonella, Campylobacter, Eimeria, Clostridium, Escherichia coli and alternate Enterobacteriaceae. As probiotics are known to help with health and well-being, one use of the disclosed probiotic composition 10 is to improve overall flock health. Improvement of overall flock health can be measured by increased weight conversion and a decrease in mortality rates throughout the lifecycle. In one or more embodiments, the probiotic mixture 11 includes a probiotic that may be selected from a group consisting of: Aspergillus niger, Aspergillus oryzae, Bacillus amyloliquefaciens, Bacillus coagulens, Bacillus lentus, Bacillus pumilus, Bacteroides amylophilus, Bacteroides capillosus, Bacteroides ruminocola, Bacteroides suis, Bifidobacterium adolescentis, Bifidobacterium animalis, Bifidobacterium infantis, enterococcus cremorus, Enterococcus diacetylactis, Enterococcus faecium, Enterococcus intermedius, Enterococcus lactis, Enterococcus thermophilus, Lactobacillus animalis, Lactobacillus brevis, Lactobacillus bulgaricus, Lactobacillus cellobiosus, Lactobacillus curvatus, Lactobacillus delbruecki, Lactbacillus fermentum, Lactobacillus helveticus, Lactobacillus reuteri, Leuconostic mesenteroides, Pediococcus acidilactici, Pediococcus damnosus, Pediococcus pentosaceus, Propiniobacterium freudenreichii, Propiniobacterium shermanii, Rhodopseudomonas palustris, Saccharomyces cerevisiae, Lactobcillus casei, Lactobacillus rhamnosus, Lactobacillus plantarum, Lactobacillus paracasei, Lactobacillus acidophilus, Lactobacillus lactis, Bacillus subtilis, Bacillus lichenformis, Bifidobacterium bifidum, Bifidobacterium lactis, Bifidobacterium longum, and/or Bifidobacterium thermophilum and combinations thereof. In a preferred embodiment, the probiotic mixture 11 includes at least five probiotics, L. casei, L. rhamnosus, L. planitarum, L. paracasei, and/or B. lactis.

In the preferred embodiment, the probiotic mixture 11 includes a gastric-juice resistant probiotic 14. To be gastric-juice resistant, a probiotic must be viable in and capable of forming a biofilm in gastric juices of a livestock animal. The preferred gastric-juice resistant probiotic 14 can survive in and form a biofilm in gastric juices germane to poultry. In some embodiments, the gastric-juice resistant probiotic 14 can survive in the gastric juices of juvenile and/or adult poultry. In certain embodiments, the gastric-juice resistant probiotic 14 can survive in the gastric juices of pre-hatching poultry. It is desirable that one of the probiotics making up the probiotic mixture 11 can survive in, and form a biofilm in, poultry GI tracts so as in ensure the overall success of the probiotic mixture 11 in outcompeting pathogenic bacterium.

In one or more embodiments, the probiotic mixture 11 includes a heat-resistant probiotic (not shown) that can survive at temperatures up to about 180° F. (+/−10° F.). In the preferred embodiment, the probiotic mixture 11 includes at least two heat-resistant probiotics that can survive at temperatures up to about 180° F. (+/−10° F.). It is desirable that the probiotic mixture 11 includes a heat-resistant probiotic because in embodiments where the probiotic mixture 11 is incorporated into a feed pellet, the pelleting process can reach temperatures between of about 160° F. to 180° F. (+/−10° F.)

The present disclosure contemplates embodiments where one species of probiotic may fill the role of both the heat-resistant probiotic and the gastric-juice resistant probiotic 104 simultaneously. The present disclosure further contemplates embodiments where the probiotic mixture 11 is only composed of one unique species of probiotic.

In some embodiments, the probiotic composition 10 includes a prebiotic 15 that may be selected from a group consisting of plant based oils (including soybean oil, sunflower oil, coconut oil and all vegetable oils), xylo-oligosaccharides, fructo-oligosaccharides, galacto-oligosaccharides, xylan, pullulan, gentibiose, polydextrose, and lactitol and/or mixtures thereof. The ability of Lactobacillus and other probiotic species to colonize the GI tract is also dependent on the presence of key nutrients. Specifically, prebiotics that are utilized as a carbon source for the bacterial strains. These prebiotics may be broken down into short chain fatty acids to provide a mechanistic basis for outcompetition. A preferred embodiment utilizes at least a xylo-oliogsaccharide and/or fructo-oligosaccharide. It is particularly desirable to include a prebiotic 15 in the probiotic composition 10 so that the viability of the probiotics is increased upon entry into the GI track of pre-hatching and/or post-hatching poultry. In the preferred embodiment of probiotic composition 10, a prebiotic 15 is included in the composition in a total weight range from 0.0001% to 10% w/w of the final composition, most preferably the prebiotic 15 is included from 0.005% to 1% w/w of probiotic composition 10. In embodiments where the probiotic composition 10 is incorporated into an eggwash, the prebiotic 15 may be included at ranges in the final eggwash from 0.0001% to 10% w/w, most preferably the prebiotic 15 ranges in the final eggwash from 0.005% to 1% w/w. In embodiments where the probiotic composition 10 is incorporated into a final feed or drinking water source, the prebiotic 15 may be included in the final feed or water source at ranges from 0.0001% to 10% w/w, most preferably the prebiotic 15 ranges in the final feed or water source from 0.005% to 1% w/w. In one or more embodiments, the prebiotic 15 may be provided in the isolated form, such as pure fructo oligosaccharide. In other embodiments, the prebiotic 15 may be provided as a precursor material that is high in prebiotic content, such as but not limited to sugarcane or naturally occurring xylan with xylo-oligosaccharide, or chicory root and inulin for fructo-oligosaccharides.

In the preferred embodiment, the probiotic composition 10 includes a complex-starching reducing enzyme (not shown) that is capable of breaking down starches, most preferably breaking down the prebiotic(s) 15, into available nutrients that can be further processed by bacteria into antimicrobial compounds. It is desirable for the probiotic composition 10 to include a complex-starch reducing enzyme because the availability of nutrients derived from complex starches that exist in plant-based diets is limited. These starches must first be broken down by enzymes to release the nutrients and create optimal growth conditions within the GI tract. Enzymes are one way to reduce complex starches into available nutrients that can be further processed by probiotics into antimicrobial compounds. In one or more embodiments, the probiotic composition 10 includes a complex-starch reducing enzyme selected from the group consisting of α-amylase, β-amylase, esterase, α-galactosidase, β-glucosidase, hemicellulose, invertase, lactase, β-mannase, pectinase, pullulanase, xylase, cellulase, xylosidase, β-glucanase, xyloglucanase, lipase, phytase, a mixture of enzymes to release entrapped energy and nutrients, and combinations thereof. In the preferred embodiment, the complex-starch reducing enzyme present in the probiotic composition 10 ranges from 0.001% to 5%, most preferably the complex-starch reducing enzyme ranges from 0.005% to 1%. In embodiments where the probiotic composition 10 is incorporated into an eggwash, the complex-starch reducing enzyme can be included at ranges in the final eggwash from 0.001% to 5%, most preferably the complex-starch reducing enzyme ranges in the final eggwash from 0.005% to 1%. In embodiments where the probiotic composition 10 is incorporated into a final feed or water source, the complex-starch reducing enzyme can be included in the final feed or water source at ranges from 0.001% to 5%, most preferably the complex-starch reducing enzyme ranges in the final feed or water source from 0.005% to 1%. One example of a mixture of enzymes are those recovered from the growth of the fungal species Trichoderma reesei. T. reesei is a filamentous fungal species with the capability of producing over 20 different lignocellulitic enzymes at a given time.

In the preferred embodiment, the probiotic composition 10 includes an enzymatic co-factor 17. Many bacteria require enzymatic co-factors to thrive in a given environment. These enzymatic co-factors can provide a competitive edge to probiotic strains and, in particular, the probiotic strains listed in this disclosure. As such, the probiotic composition 10 can also contain an enzymatic co-factor 17 in the range from 0.001 to 1.0% w/w. Optimally, the enzymatic co-factor 17 may be included at a range from 0.005% to 0.5% w/w. In embodiments where the probiotic composition 10 is incorporated into an eggwash, the enzymatic co-factor 17 can be included at ranges in the final eggwash from 0.001% to 1.0% w/w, most preferably the enzymatic co-factor 17 ranges in the final eggwash from 0.005% to 0.5% w/w. In embodiments where the probiotic composition 10 is incorporated into a final feed or water source, the enzymatic co-factor 17 can be included in the final feed or water source at ranges from 0.001% to 1.0% w/w, most preferably the enzymatic co-factor 17 ranges in the final feed or water source from 0.005% to 0.5% w/w. In some embodiments, the enzymatic co-factor 17 can be selected from a group consisting of: fat soluble vitamins (such as but not limited to A, D, E, and K), water soluble vitamins (such as but not limited to B₁, B₂, B₃, B₆, B₁₂, biotin, folic acid, pantothenic acid, and C) and divalent cations (such as but not limited to magnesium, manganese, calcium, beryllium, barium, strontium, cobalt, copper, nickel, iron, and cadmium or salts thereof), and/or mixtures thereof.

In one or more embodiments, the probiotic composition 10 includes a pH modifier 16. In some embodiments, the pH modifier 16 is selected from a group consisting of citrate (and salts thereof), acetate (and salts thereof), fumaric acid (and salts thereof), glutamic acid (and salts thereof), phosphoric acid (and salts thereof), and/or mixtures thereof. In order to provide optimal growth within the GI tract, the pH modifier 16 may also be included in the formulation in the range from 0.001 to 1.0% w/w. Optimally, the pH modifier 16 may be included at a range from 0.005% to 0.5% w/w. In embodiments where the probiotic composition 10 is incorporated into an eggwash, the pH modifier 16 may be included at ranges in the final eggwash from 0.001% to 1.0% w/w, most preferably the pH modifier 16 ranges in the final eggwash from 0.005% to 0.5% w/w. In embodiments where the probiotic composition 10 is incorporated into a final feed or water source, the pH modifier 16 can be included in the final feed or water source at ranges from 0.001% to 1.0% w/w, most preferably the pH modifier 16 ranges in the final feed or water source from 0.005% to 0.5% w/w.

Various application methods utilizing probiotic composition 10 to prevent pathogen growth in poultry GI tracts are considered within the scope of the present disclosure. The probiotic composition 10 may be incorporated into the GI tracts of poultry by including the probiotic composition 10 in food or water ingested by the poultry or directly spraying the poultry. The probiotic composition 10 may also be incorporated into the GI tracts of poultry by applying the probiotic composition 10 as an eggwash to eggs prior to or during incubation (best seen in FIG. 1). These methods of use will be discussed in detail in the proceeding paragraphs and examples.

The present disclosure contemplates an ingestion method for impregnating a poultry GI tract with probiotic composition 10. The ingestion method comprises providing the probiotic composition 10. In one or more embodiments, the ingestion method comprises providing the probiotic composition 10 as a concentrated powder and diluted on site to the indicated CFU per ton of feed. In alternative embodiments, the ingestion method comprises providing the probiotic composition 10 as a pre-diluted solution with active bacteria to a feed mill or poultry housing unit. The ingestion method further comprises applying the probiotic composition 10 to a substance that is ingestible by poultry. In certain embodiments, the probiotic composition 10 may be applied to the feed, as a mash prior to pelleting and/or as a post-pelleting spray applied wet or dry. In some embodiments, the probiotic composition 10 may be applied to a drinking water source as a water additive. In alterative embodiments, the probiotic composition 10 may be applied directly to poultry as a poultry direct spray so that the animal may ingest it when preening or by swallowing spray that enters the interior of their beak. Regardless of the medium by which probiotic composition 10 is ingested by the animal, once ingested, the probiotic composition 10 will establish a biofilm in the GI tract of the animal that will exclude pathogenic bacteria.

In certain embodiments where the probiotic composition 10 is applied to feed, the components of probiotic composition 10 in the final feed preferably exist in a range of CFU from 10¹ to 10¹⁵ per ton of feed. The more preferred concentration is 10² to 10¹² CFU per ton of feed. The most preferred application rate is 10³ to 10¹⁰ CFU per ton of feed. In other embodiments, the components of the probiotic composition 10 in the final feed exist in a range of CFU from 10² to 109 CFU per ton of feed.

In certain embodiments, where the probiotic composition 10 is applied to feed as a mash prior to pellet, the probiotic composition 10 includes a heat-resistant probiotic that can survive at temperatures up to about 180° F. (+/−10° F.) and/or survive a pelleting process. In a preferred embodiment, the probiotic composition 10 includes at least two heat-resistant probiotics that can survive at temperatures up to about 180° F. (+/−10° F.) and/or survive the pelleting process. Once probiotic composition 10 is formulated into the feed, at the required concentration, at least one of the bacterial species described above will survive the pelleting process of 160 to 180° F. with shearing from a die cutter.

The present disclosure also contemplates a pre hatching uptake method for impregnating a poultry GI tract 104 of a developing embryo 103 with probiotic composition 10. The pre hatching uptake method comprises providing the probiotic composition 10 in a liquid solution, an “eggwash.” The probiotic composition 10 is at a concentration ranging from 10⁵ CFU/mL to 10⁹ CFU/mL, preferably the liquid solution is at concentration ranging from 10⁶ CFU/mL to 107 CFU/mL. In other embodiments, the probiotic composition 10 is at a concentration ranging from 10² CFU/mL to 10⁹ CFU/mL. The pre hatching uptake method further comprises spraying the probiotic composition 10 onto an exterior surface 102 of an eggshell 101. In the preferred embodiment, the sprayer sprays approximately 1+/−0.02 mL of probiotic composition 10 onto the egg 100 at a 450 angle from approximately 12 inches (+/−2 inches) from the exterior surface 102 of the egg 100. In some embodiments, the pre hatching uptake method further comprises air drying the egg and in other embodiments moving air or heat may be applied to accelerate drying. In the preferred embodiment, the pre hatching uptake method comprises incubating the sprayed egg. After the probiotic composition 10 is deposited on the exterior surface 102 of the egg 100, the probiotic composition 10 traverses the eggshell 102 to the albumin 105 and/or egg yolk 106 and then the developing embryo 103 will ingest the probiotic composition 10. The ingested probiotic composition 10 will then establish a biofilm in the GI tract 104 of the developing embryo 103 that will exclude pathogenic bacteria.

### Example 1: Antagonistic Activity Against Pathogenic Bacteria is Species Specific

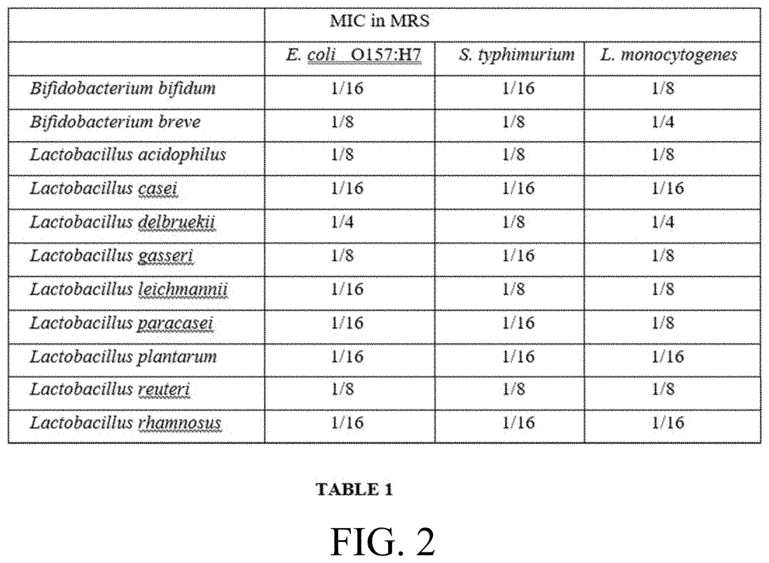

There is an inhibitory effect of potential probiotic strains against E. coli, S. typhimirium, and L. monoctyogenes. Briefly, bacteria were grown in De Man, Rosa and Sharpe (MRS) broth and the supernatant isolated. The minimum inhibitory concentration (MIC) of the supernatant was evaluated against the three listed pathogens. The lower the number the higher the antagonistic effect of the probiotic supernatant. As noted in the data, some strains and species have improved antagonistic activity when compared to others as seen in Table 1 of FIG. 2.

### Example 2: Acid and Bile Tolerance is Species Specific

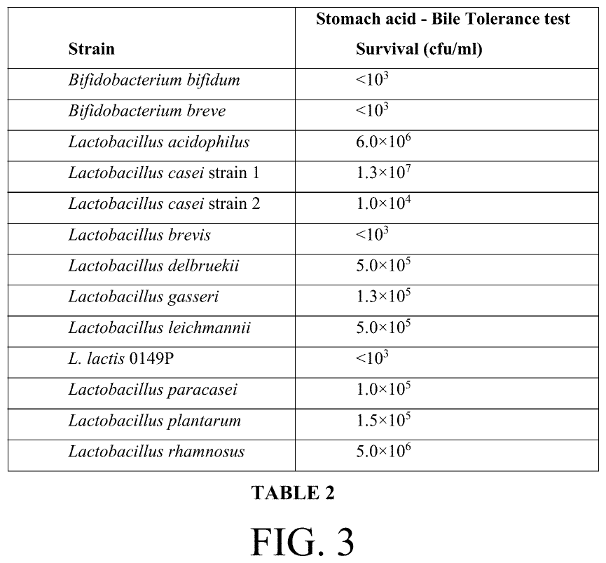

These strains were subjected to exposure to bile and stomach acids. Following exposure, the resulting colonies were counted. Those exhibiting higher CFU counts demonstrate an increased resistance to stomach acid and bile salts. As demonstrated in Table 2 of FIG. 3, bile and acid sensitivity is uniquely strain specific.

### Example 3: Planktonic Out Competition of Salmonella

Typical outcompetition focuses on planktonic, free swimming, cells of two different species competing for the same nutrients. To compare the ability of the selected probiotic strains to outcompete Salmonella, each strain was allowed to form a biofilm first. Briefly, the probiotic strains were added separately or as a mixture in MRS broth supplemented with 10% heat treated egg yolk into 96 well dishes and incubated anaerobically at 37° C. for 18-24 hours. Once a biofilm was formed, the media was removed and replaced with MRS broth+10% yolk that had been inoculated with Salmonella enterica serovar newport. This was incubated anaerobically at 37° C. for 18-24 hours. The resulting planktonic media was recovered and enumerated on Brilliant Green agar to select for Salmonella.

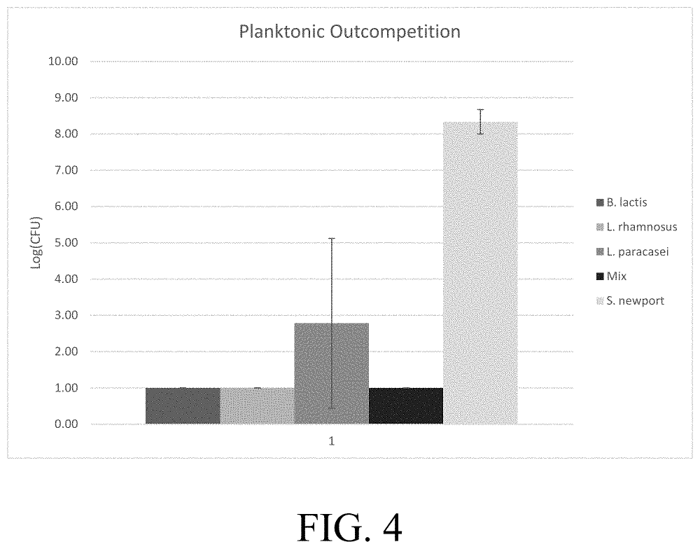

B. lactis and L. rhamnosus were able to reduce Salmonella in the planktonic state below the limit of detection, a comparative >7 log reduction. L. paracasei had increased variability with the reduction but still performed at a >5 log reduction level as shown in FIG. 4. This indicates an ability to out compete for all selected strains in the planktonic state.

### Example 4: Biofilm Out Competition of Salmonella

Biofilm exclusion is the process by which one bacterial species is already established on a surface and a second species then enters the environment. The biofilm of the first species prevents the second species from adhering to the surface, thereby excluding the second species. In order to investigate biofilm exclusion, each strain was allowed to form a biofilm first. Briefly, the probiotic strains were added separately or as a mixture in MRS broth supplemented with 10% heat treated egg yolk into 96 well dishes and incubated anaerobically at 37° C. for 18-24 hours. Once a biofilm was formed, the media was removed and replaced with MRS broth+10% yolk that had been inoculated with Salmonella enterica serovar newport. This was incubated anaerobically at 37° C. for 18-24 hours. The media was removed and replaced with 100 μL of phosphate buffered saline (PBS). The biofilm in the well was scraped into the PBS and enumerated on Brilliant Green Agar to distinguish Salmonella.

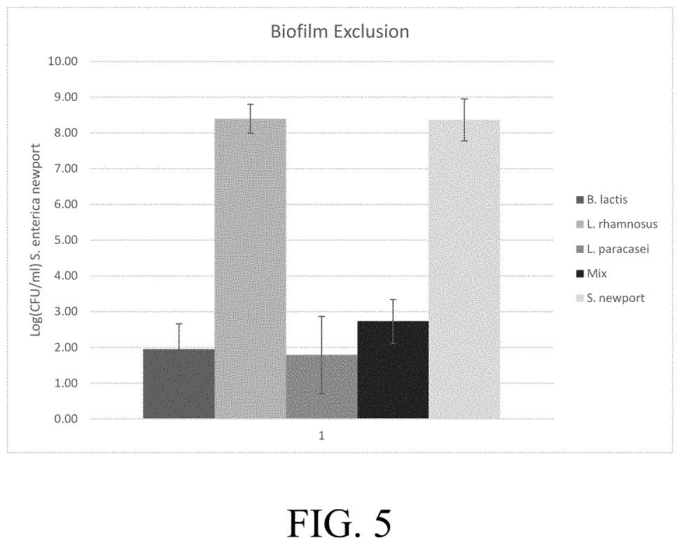

As demonstrated in FIG. 5, B. lactis and L. paracasei provides a complete reduction below the lower limit of detection for Salmonella, a >6 log reduction. Interestingly, while L. rhamnosus was able to effectively outcompete Salmonella it was not able to exclude. This indicates that a mixture of probiotic strains is optimal for efficient out competition of target pathogens.

### Example 5: Feed Composition is Critical for Gram Negative Outcompetition

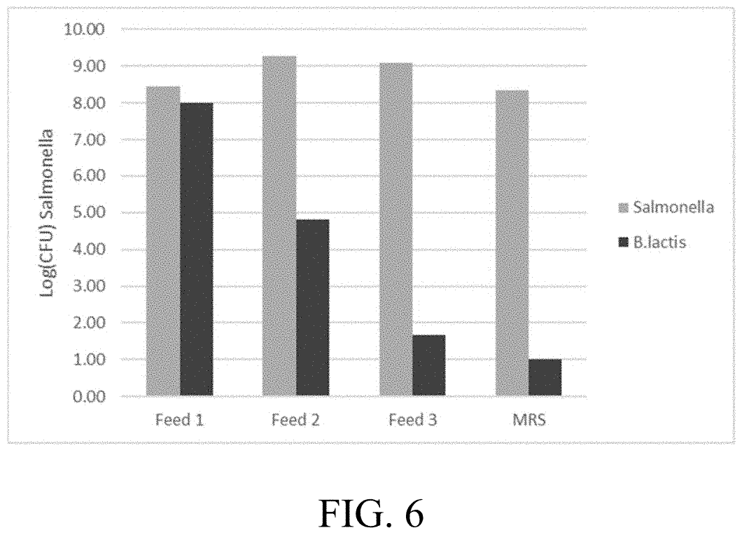

The data presented in examples 3 and 4 were conducted in laboratory based media developed in the 1960s by De Man, Rosa and Sharpe (MRS). MRS media components are not reflective of animal feed components. In fact, the fast majority of media components are not allowed to be fed to animals. Therefore it is important to understand the ability of traditional animal feed components to support the outcompetition ability of the identified bacterial species. To this end, industry standard feed was hydrated and exposed to digestive enzymes. Following digestion the soluble fraction was removed from the solid fraction and retained for testing. The soluble fraction of two different animal feeds were utilized for both planktonic outcompetition and biofilm exclusion. Surprisingly, the bacteria demonstrating the highest activity in MRS are rendered ineffective in traditional animal feed. Additionally, Feed 3 is able to outperform Feed 1 demonstrating that feed composition is critical for outcompetition metrics. FIG. 6 shows feed composition plays a critical role in outcompetition.

### Example 6: Feed Composition Alone Reduces Growth of Poultry Pathogens

Based on the findings demonstrated in Example 5 it became imperative to formulate a feed that consistently demonstrates outcompetition of pathogens in the assays previously described herein. In this example, the feed alone was assessed for the ability to support pathogen growth, no probiotic was added within this example. Feed was processed as described in Example 5 and then inoculated with the Salmonella enteritidis to represent gram negative poultry pathogens of concern or Enterococcus cecorum to represent gram positive poultry pathogens of concern. The samples were incubated at 37° C. for 18 to 24 hours. Following incubation, S. enteritidis samples were enumerated on Brilliant Green agar to select for only Salmonella species growth.

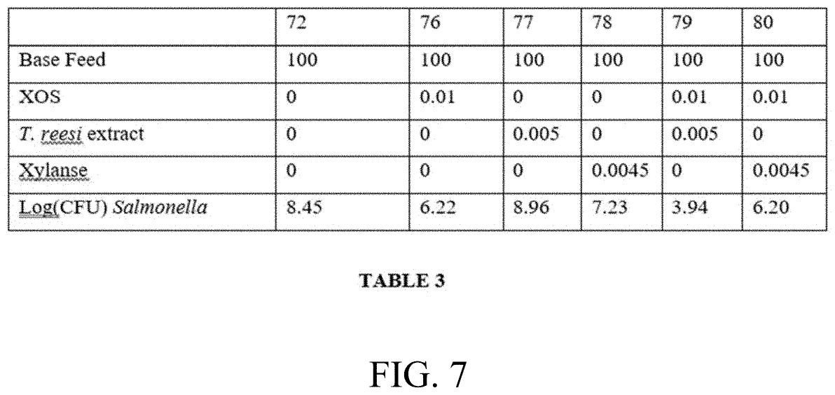

Table 3 shown in FIG. 7 shows feed composition impacts ability of pathogens to grow without introduction of a probiotic. Each represents a gram weight addition.

As demonstrated, the base feed (formula 72) supports the growth of gram negative pathogens. The feed composition itself can be altered to include prebiotics and enzymes (Formula 79) that decrease the ability of pathogens to survive. While the prebiotic alone (Formula 76) does decrease the ability of Salmonella to survive it is not to the same level as that of the combinatorial use of enzymes and prebiotics observed with Formula 79. In fact, enzymes alone have a negligible impact on pathogen survival allowing numbers to reach similar levels to that of the base feed alone (compare Formula 72 with Formula 77 or 78).

### Example 7: Feed Composition Promotes Outcompetition in the Presence of a Probiotic

Example 6 was repeated in the presence of a probiotic strain (B. lactis) to determine the influence of probiotics on further reducing the pathogen concentration in the determined feed compositions. The same protocol was used as described in Example 6 with the addition of 10⁴ CFU/ml of B. lactis to each feed composition.

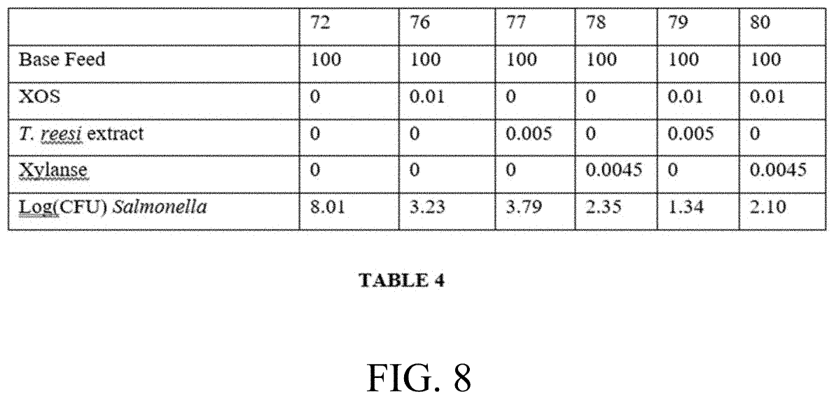

Table 4 in FIG. 8 shows feed composition including probiotics enhances the ability to reduce pathogens of concern. Each represents a gram weight addition.

As demonstrated, the addition of B. lactis to the base feed composition had no impact on the ability of B. lactis to outcompete Salmonella. However, in combination with a prebiotic or enzyme, B. lactis was able to provide substantial improvement of Salmonella outcompetition, between a 2 to 5 log reduction was observed when comparing feed compositions lacking B. lactis(Compare Table 3 and 4). Interestingly, the combination of B. lactis, a prebiotic, and an enzyme (Formula 79 and 80 from Table 4) provide the lowest total Salmonella in the feed demonstrating a possible synergistic activity.

### Example 8: Feed Composition Promotes Outcompetition of Gram Positive Species

Enterococcus cecorum is a gram positive emerging poultry pathogen that is a GI tract commensal. It can invade cardiac and thoracic tissue causing significant morbidity and mortality within a poultry flock. To determine if the same feed formulation (Formula 79 from Table 3 and 4) provided outompetition of gram positive bacteria a Zone of Inhibition (ZOI) assay was performed. E. cecorum was grown on bile esculin azide agar (BEA). When grown on BEA, Enterococcus will hydrolyze esculin giving the media a brown to black color. The absence of brown color indicates inhibition of Enterococcus.

Cultures of E. cecorum isolates were grown in sterile BHIB overnight at 36° C. 1 ml of the overnight culture was transferred into a new sterile tube. 10 mL of sterile, molten Bile Esculin Azide Agar was added to the tube and inverted to mix. This was poured into a 100 mm sterile petri dish and allowed to solidify. Following solidification, the large end of a sterile 1 mL pipette tip was utilized to bore a hole in the agar. The bore was removed, leaving a hole in the agar.

After making the feed according to the composition listed, 10 g of feed was added to 90 g of water and autoclaved. Following autoclave, the resulting mixture was digested with a mixture of trypsin and chymotrypsin at 37° C. overnight. Enzymes were inactivated at 60 C for 10 minutes. The broth section of the media was removed from the remnant feed by centrifugation. Broth was stored at 4° C. until ready to use. 1 mL of the resulting feed was removed into a sterile tube and warmed at 60° C. for ≥10 minutes. Once warm, ˜20 μL of 10% molten agar was added to the tube. 100 μl was then pipetted into the hole of the corresponding BEA agar. This was repeated for each strain and media type.

Once all sections were filled and solidified, the plate was incubated at 36° C. overnight. After incubation the clear zones were measured and pictures were taken.

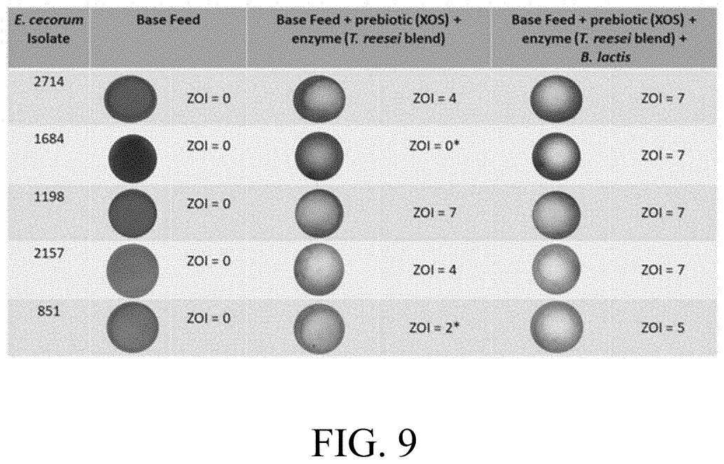

FIG. 9 shows feed composition along with probiotics promotes successful outcompetion of gram positive species.

As shown in FIG. 9, the addition of prebiotic and enzyme to the base feed enhance the activity against the gram positive pathogen but does not entirely clear the pathogen of concern. When the prebiotic and enzyme feed is utilized with a probiotic species, such as B. lactis, there is a complete Zone of Inhibition. This is true for all E. cecorum isolates tested to date.

### Example 9: In Vivo Trial

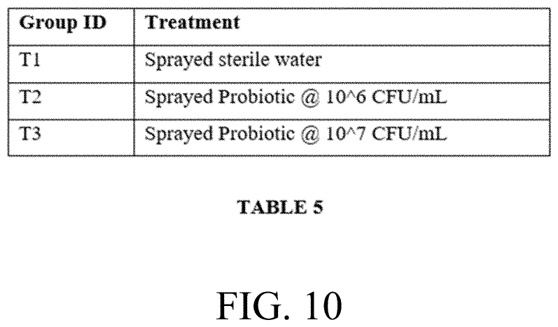

Three hundred twenty Ross x Ross broiler eggs were obtained from University of Georgia breeder flock. The eggs were split into three different treatment groups with 120 eggs per group as defined in the Table 5 of FIG. 10.

The flats of eggs for each treatment were weighed both pre and post application to determine volume deposition per egg. The sprayer was held to generate 1+/−0.02 ml onto each egg. In trials, this approximated the sprayer being held at a 450 angle approximately 12 inches from the egg surface. Eggs were treated just prior to egg set, allowed to dry and immediately placed in the incubator designated for that treatment. At the indicated time points the jejunum was sampled for both microbiome contents and then isolated in formalin for downstream histology.

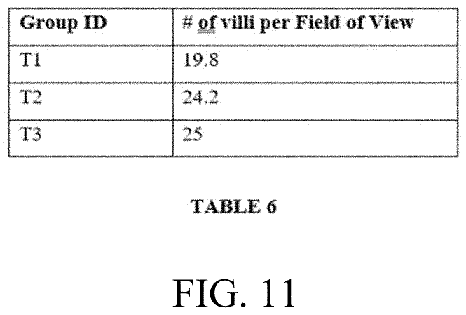

Following treatment, eggs were incubated for 18 days and then set for hatch. At day 18 of incubation, the jejunum of 5 developing embryos from each group were sampled. Of these, the samples from T2 and T3 demonstrated a positive change in the jejunum architecture, see Table 6 in FIG. 11. The number of villi per field of view increased when compared to the T1 control group (p<0.05). The ability to influence the GI track with an eggwash has never been demonstrated.

Additionally, there is stability in GI track colonization because the probiotic species was able to be detected in the jejenum even 4 days post hatch. Colonization rate was dose dependent as 5 out of 5 animals were colonized in T3 but only 3 out of 5 animals in the T2 group.

The illustrations and examples provided herein are for explanatory purposes and are not intended to limit the scope of the appended claims.

I claim:

1. A probiotic composition for reducing pathogenesis in livestock comprising:
a probiotic mixture (11);
a prebiotic (15), said prebiotic (15) functioning as a carbon source for said probiotic;
a complex-starch catalyzing enzyme; said complex-starch catalyzing enzyme configured to break down the complex starches into additional prebiotics;
wherein the probiotic composition (10) forms a biofilm within a GI tract of livestock that excludes pathogenic bacterium and facilitates the outcompetition of pathogenic bacterium.

2. The probiotic composition of claim 1, wherein said probiotic mixture (11) comprising a gastric-juice resistant probiotic (14) that is capable of surviving the gastric juices of pre- and/or post-hatching poultry and establishing a biofilm within pre- and/or post-hatching poultry GI tracts.

3. The probiotic composition of claim 2, wherein said probiotic mixture (11) further comprising a heat-resistant probiotic that is capable of surviving a temperature of up to 180° F. and establishing a biofilm after being exposed to a temperature of up to 180° F., said heat-resistant probiotics also capable of surviving a pelleting process.

4. The probiotic composition of claim 3, wherein said probiotic mixture (11) includes two heat-resistant probiotics.

5. The probiotic composition of claim 3, wherein the probiotic mixture (11) is selected from a group consisting of: Lactobacillus and Bifidobacterium, and mixtures thereof.

6. The probiotic composition of claim 3, wherein the probiotic composition (10) further comprises a pH modifier (16), said pH modifier (16) is selected from a group consisting of citrate (and salts thereof), acetate (and salts thereof), fumaric acid (and salts thereof), glutamic acid (and salts thereof), phosphoric acid (and salts thereof), and mixtures thereof.

7. The probiotic composition of claim 3, wherein the probiotic composition (10) further comprises an enzymatic co-factor (17), said enzymatic co-factor (17) is be selected from a group consisting of: fat soluble vitamins, water soluble, and divalent cations, and mixtures thereof.

8. The probiotic composition of claim 3, wherein the complex-starch reducing enzyme is selected from the group consisting of: α-amylase, β-amylase, esterase, α-galactosidase, β-glucosidase, hemicellulose, invertase, lactase, β-mannase, pectinase, pullulanase, xylase, cellulase, xylosidase, β-glucanase, xyloglucanase, lipase, phytase, or a mixture of enzymes to release entrapped energy and nutrients, and mixtures thereof.

9. The probiotic composition of claim 3, wherein the prebiotic (15) is selected from a group consisting of plant based oils, xylo-oligosaccharides, fructo-oligosaccharides, galacto-oligosaccharides, xylan, pullulan, gentibiose, polydextrose, and lactitol and mixtures thereof.

10. An ingestion method for impregnating a livestock GI tract with the probiotic composition of claim 3 comprising: providing the probiotic composition (10) and applying the probiotic composition (10) to a substance that is ingestible by the livestock.

11. The ingestion method of claim 10, wherein the probiotic composition (10) is provided as a concentrated powder, wherein the ingestion method further comprises diluting the concentrated powder to a predetermined amount of CFU per ton of a feed.

12. The ingestion method of claim 10, wherein the probiotic composition (10) is provided as a pre-diluted solution.

13. The ingestion method of claim 10, wherein the probiotic composition (10) is added to a pre-pelleting feed, formed from the pelleting process, as a mash.

14. The ingestion method of claim 13, wherein the probiotic composition (10) includes at least two heat-resistant probiotics.

15. The ingestion method of claim 14, wherein the probiotic composition (10) is present in a post-pelleting feed at a concentration ranging from 10³ to 10¹⁰ CFU per ton of the post-pelleting feed.

16. The ingestion method of claim 10, wherein the probiotic composition is added to a post-pelleting feed as a wet or dry spray.

17. The ingestion method of claim 10, wherein the probiotic composition is applied to a water source as a water additive.

18. A probiotic feed additive comprising:
a probiotic mixture (11) comprising:
At least one gastric-juice resistant probiotic (14) capable of surviving in and forming a biofilm in a GI environment, a prebiotic (15) for increasing the viability of the probiotic mixture;
a pH modifier (16);
a co-factor (17);
wherein, the probiotic feed additive is ingestible by a livestock animal so that a GI tract microbiome is established that outcompetes pathogens.

19. The probiotic feed additive of claim 18, wherein said probiotic mixture (11) is selected from a group consisting of Lactobacillus and Bifidobacterium, and mixtures thereof.

20. The probiotic feed additive of claim 18, wherein said co-factor (17) is selected from a group consisting of: fat soluble vitamins, water soluble vitamins, and divalent cations, and mixtures thereof; wherein said pH modifier (16) is selected from a group consisting of: citrate and salts thereof, acetate and salts thereof, fumaric acid and salts thereof, glutamic acid and salts thereof, phosphoric acid and salts thereof, magnesium sulfate, and manganese sulfate, and mixtures thereof; wherein said prebiotic (15) is selected from a group consisting of: plant-based oils, xylo-oligosaccharides, fructo-oligosaccharides, galacto-oligosaccharides, xylan, pullulan, gentibiose, polydextrose, and lactitol and mixtures thereof.
